(19) United States
(12) Patent Application Publication
(10) Pub. No.: US 2025/0272831 A1
(43) Pub. Date: Aug. 28, 2025

(54) AUTOMATED METHODS FOR DETERMINING FIBROGLANDULAR DENSITY ON MAMMOGRAMS

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Krishna Juluru, New York, NY (US); Krishna Nand Keshava Murthy, New York, NY (US); Amin El-Rowmeim, New York, NY (US); Pierre El-Najjar, New York, NY (US); Hao-Hsin Shih, New York, NY (US); Gordon Watt, New York, NY (US)

(21) Appl. No.: 18/856,763

(22) PCT Filed: Apr. 13, 2023
(86) PCT No.: PCT/US2023/018551

§ 371 Date: Oct. 14, 2024

Related U.S. Application Data
(60) Provisional application No. 63/331,578, filed on Apr. 15, 2022.

(51) Int. Cl. G06T 7/00; A61B 6/00; A61B 6/50; G06T 7/11; G06V 10/25; G06V 10/764; G06V 10/774; G16H 50/30

(52) U.S. Cl. CPC G06T 7/0012; A61B 6/502; A61B 6/5217; G06T 7/11; G06V 10/25; G06V 10/764; G06V 10/774; G16H 50/30; G06T 2207/20081; G06T 2207/30068; G06T 2207/30096; G06V 2201/03

(57) ABSTRACT

Presented herein are systems and methods of determining density values from mammograms. A computing system may identify a first mammogram of a first breast region of a first subject. The first mammogram may have a first region of interest (ROI) corresponding to a first dense area of the first breast region. The computing system may apply the first mammogram to a machine learning (ML) model to generate a first segmentation map identifying the first ROI within the first mammogram. The computing system may determine a density value for the first dense area of the first breast region based on the first segmentation map.

### CROSS-REFERENCE TO RELATED APPLICATIONS

This application claims the benefit of and priority to U.S. Provisional Patent Application No. 63/331,578, filed Apr. 15, 2022, the contents of which are incorporated herein by reference in its entirety.

### TECHNICAL FIELD

The present technology relates generally to methods and systems for automated determination of the density or amount of fibroglandular tissue (FGT) from mammograms obtained from subjects, and the application of machine learning to categorize samples based on mammographic density to assess the risk for breast cancer.

### BACKGROUND

The following description of the background of the present technology is provided simply as an aid in understanding the present technology and is not admitted to describe or constitute prior art to the present technology.

Mammographic density is an established predictor of breast cancer risk that is widely used in clinical practice (1,2). Mammographic density is typically assessed subjectively by a breast radiologist who categorizes the density, or “amount” of fibroglandular tissue (FGT), into one of four qualitative categories according to the Breast Imaging-Reporting and Data System (BI-RADS): almost entirely fatty; scattered fibroglandular density; heterogeneously dense; or extremely dense. In clinical practice, density is estimated subjectively using BI-RADS, which has unacceptably high intra-and inter-observer variability, precluding reproducible risk prediction.

For example, in one study of radiologists in the United States and United Kingdom, inter-observer agreement (Fleiss's κ) ranged from 0.42 (poor) to 0.84 (substantial) for individual BI-RADS categories (3); while in another study of experienced radiologists, intra-observer agreement (Cohen's κ) ranged from 0.50 (poor) to 0.81 (substantial) (4). To fully realize the potential of mammographic density for personalized breast cancer risk assessment, there is a critical need to make the measurement quantitative, accurate, and reproducible.

### SUMMARY

Aspects of the present disclosure are directed to systems and methods of determining density values from mammograms. A computing system may identify a first mammogram of a first breast region of a first subject. The first mammogram may have a first region of interest (ROI) corresponding to a first dense area of the first breast region. The computing system may apply the first mammogram to a machine learning (ML) model to generate a first segmentation map identifying the first ROI within the first mammogram. The ML model may be established using a training dataset comprising a plurality of examples. Each of the plurality of examples may include (i) a respective second mammogram of a second breast region of a corresponding second subject and (ii) a respective second segmentation map identifying a second ROI in the respective second mammogram corresponding to a second dense area of the second breast region. The computing system may determine a density value for the first dense area of the first breast region based on the first segmentation map. The computing system may store, using one or more data structures, an association between the first subject and the density value.

In some embodiments, the computing system may classify the first subject into one of a plurality of risk levels each associated with a likelihood of occurrence of breast cancer based on the density value for the first dense area of the first breast region. In some embodiments, the computing system may categorize the first dense area of the first breast region into one of a plurality of density types based on a ratio of a first portion of the first segmentation map corresponding to the first ROI and a second portion of the first segmentation map outside the first ROI.

In some embodiments, the computing system may provide information for presentation based on the association between the first subject and the density value. In some embodiments, the breast cancer may be HER2-positive breast cancer, estrogen receptor-positive breast cancer, progesterone receptor-positive breast cancer, or triple negative breast cancer.

In some embodiments, one or more of a radiation therapy, immunotherapy, chemotherapy or surgery may be administered to the first subject, when the density value of the first subject is elevated relative to a predetermined threshold. In some embodiments, the predetermined threshold may be based on a plurality of density values from a corresponding plurality of control subjects without breast cancer. In some embodiments, the computing system may receive, from a mammograph device, the first mammogram of the first breast region of the first subject (i) prior to diagnosis of breast cancer in the first subject or (ii) after treatment of the breast cancer in the first subject.

In some embodiments, the computing system may determine the density value based on a ratio of a first portion of the first segmentation map corresponding to the first ROI and a second portion of the first segmentation map outside the ROI. In some embodiments, the density value may comprise a fibroglandular density value selected from among entirely fatty, scattered fibroglandular density, heterogeneously dense, or extremely dense.

Aspects of the present disclosure are directed to systems and methods of training models to determine density values from mammograms. A computing system may identify a training dataset comprising a plurality of examples. Each example of the plurality of examples may include (i) a respective first mammogram of a first breast region of a corresponding first subject and (ii) a respective first segmentation map identifying a region of interest (ROI) in the first second mammogram corresponding to a first dense area of the first breast region. The computing system may apply the respective first mammogram from each example of the plurality of examples to a machine learning (ML) model comprising a set of weights to generate a corresponding second segmentation map identifying the ROI in the first mammogram to be used to determine a density value for the first dense area of the first breast region. The computing system may determine a loss metric based on a comparison between (i) the respective first segmentation map of each example of the plurality of examples of the training dataset and (ii) the corresponding second segmentation map generated from the respective first mammogram associated with the respective first segmentation map. The computing system may update at least one of the set of weights of the ML model using the loss metric.

In some embodiments, the computing system may store the set of weights for the ML model to use to apply to a third mammogram of a second breast region of a second first subject acquired via a mammograph device to generate a third segmentation map identifying a second ROI within the third mammogram. In some embodiments, the computing system may select, from the plurality of examples of the training dataset, a subset of examples for training the ML model.

In some embodiments, the computing system may determine a similarity value as a function of the (i) the respective first segmentation map of each example of the plurality of examples of the training dataset and (ii) the corresponding second segmentation map generated from the respective first mammogram. In some embodiments, the respective first mammogram of the first breast region of the first subject in each of the plurality of examples of the training dataset may be obtained at one of (i) prior to diagnosis of breast cancer in the first subject or (ii) after treatment of the breast cancer in the first subject.

In some embodiments, the breast cancer may be HER2-positive breast cancer, estrogen receptor-positive breast cancer, progesterone receptor-positive breast cancer, or triple negative breast cancer. In some embodiments, one or more of a radiation therapy, immunotherapy, chemotherapy or surgery may be administered to the first subject, when the density value of the first subject is elevated relative to a predetermined threshold. In some embodiments, the predetermined threshold may be based on a plurality of density values from a corresponding plurality of control subjects without breast cancer.

In some embodiments, the respective first segmentation map in each of the plurality of examples of the training dataset may be generated using a thresholding function applied to the respective first mammogram of the first breast region of the corresponding first subject. In some embodiments, the density value may comprise a fibroglandular density value selected from among entirely fatty, scattered fibroglandular density, heterogeneously dense, or extremely dense.

### BRIEF DESCRIPTION OF THE DRAWINGS

FIG. 1 depicts a block diagram of a system for determining density values of breasts from mammograms in accordance with an illustrative embodiment.

FIG. 2 depicts a block diagram of a process for training a density prediction model in a system for determining density values of breasts from mammograms in accordance with an illustrative embodiment.

FIG. 3 depicts a block diagram of an architecture for a density prediction model in a system for determining density values of breasts from mammograms in accordance with an illustrative embodiment.

FIG. 4A depicts a block diagram of an architecture of a transform block in a density prediction model of the system for determining density values of breasts in accordance with an illustrative embodiment.

FIG. 4B depicts a block diagram of an architecture of a set of transform layers in a transform block in a density prediction model of the system for determining density values of breasts in accordance with an illustrative embodiment.

FIG. 5 depicts a block diagram of a process for applying a density prediction model to acquired mammograms in a system for determining density values of breasts from mammograms in accordance with an illustrative embodiment.

FIG. 6 depicts a flow diagram of a method of determining density values of breasts from mammograms in accordance with an illustrative embodiment.

FIG. 7 depicts a flow diagram of a method of training models to determine density values of breasts from mammograms in accordance with an illustrative embodiment.

FIG. 8 depicts block diagram of a server system and a client computer system in accordance with an illustrative embodiment.

FIG. 9 shows the architecture of the U-net algorithm used to estimate mammographic dense area based on CUMULUS ground truth.

FIGS. 10A-10C show the comparison of dense area estimated by the intensity-thresholding software, CUMULUS, and the U-net segmentation algorithm in the holdout test set of mammograms (n=168). FIG. 10A: Histogram of the distribution of dense pixels identified using Cumulus; FIG. 10B: histogram of the distribution of dense pixels identified by the U-net MDA program; FIG. 10C: scatter plot of number of dense pixels estimated by Cumulus and the U-net MDA program.

FIG. 11 shows the histograms of the distributions of dice (left) and Jaccard (right) scores of the predicted dense area for the U-Net MDA program relative to the ground truth defined by the intensity-thresholding software, CUMULUS.

FIG. 12 shows the precision-recall curve for the overlap of dense area calculated by the U-net algorithm and dense area determined by Cumulus.

FIGS. 13A and 13B each show examples of the U-net MDA estimates on pre-treatment digitized mammograms. The left column shows the breast image (with background removed) and the ground truth segmentation contour (red) of the dense region. The middle column shows the ground truth dense region contour (red) as well as the mask (yellow). The right column shows an exemplary predicted segmentation heat map [0,1] and the ground truth contour (red) for comparison.

FIG. 14: Multivariable regression models of CBC case-control status using (A) the U-Net dense area measure disclosed herein and (B) the standard Cumulus measure of mammographic dense area, in the holdout set of mammograms in the WECARE Study. Abbreviations. CBC, contralateral breast cancer; OR, odds ratio; SE, standard error; CI, confidence interval. ^{a} Dense area in pixels, log-transformed; ^{b} OR per adjusted standard deviation of the dense area measure. Estimated in multivariable logistic regression model adjusting for estrogen receptor status of the first primary breast cancer (Negative/Positive); age at time of diagnosis of the first breast cancer; chemotherapy and radiation therapy for treatment of the first breast cancer; at-risk time for CBC; race/ethnicity (non-Hispanic White vs. Other); and recruitment cancer registry; ^{c} Automatic measurement of mammographic dense area using a convolutional neural network algorithm based on the U-Net architecture. Algorithm was trained on an independent set of mammograms from the same study.

FIG. 15: Characteristics of WECARE Study participants with mammograms selected for the training, validation, and holdout test sets used to develop the U-net algorithm.

### DETAILED DESCRIPTION

It is to be appreciated that certain aspects, modes, embodiments, variations and features of the present methods are described below in various levels of detail in order to provide a substantial understanding of the present technology.

The U-net (13), a fully convolutional Neural Network architecture (12) for biomedical image segmentation tasks, has an encoder-decoder structure, where the encoder contains convolution, non-linearity, and pooling layers to output a low-dimensional representation of the high-dimensional input image; while the decoder takes the low-dimensional representation and up-samples using a symmetric expanding path to produce an output image the same size as the high-dimensional input. Additionally, the U-net integrates skip connections between corresponding encoding and decoding layers, combining the contextual information from the low-dimensional encoded features and spatial information from the skip connections to produce the final output map (i.e., end-to-end optimization). This approach has been shown to be efficient and accurate for segmentation of biomedical images (17,18).

The present disclosure provides an automatic mammographic dense area (MDA) segmentation algorithm (measure of breast density) for the unaffected breast using a fully convolutional neural network (FCNN) (12) approach based on the U-net architecture. MDA ground truth was assessed for each unaffected breast using the semi-automated thresholding software, CUMULUS. The images were split into training (70%), validation (15%), and holdout test (15%) sets for training, hyperparameter tuning, and evaluation of performance, respectively. The case-control association between contralateral breast cancer (CBC) and mammographic dense area (MDA) measured by U-net and the Cumulus gold standard was also assessed for 51 CBC cases and 49 unilateral breast cancer (UBC) controls with mammograms taken prior to treatment for their first breast cancer. On the holdout test set, the median Dice score was 0.91, and the average precision (weighted at each intensity level) was 0.98. The intraclass correlation coefficient for the ground truth and U-net MDA was 0.92 (95% CI 0.90-0.94). In the case-control analysis, CBC had a stronger association with U-net MDA measure (OR 1.77, 1.15-2.88) than the Cumulus MDA measure (OR 1.48, 95% CI 1.00-2.27). Accordingly, the present disclosure provides a highly accurate method of automated breast density estimation that provides discrimination of breast cancer at least as well as established gold standard measures. By eliminating unpredictable variability in clinical breast density estimation, this method has the potential to improve breast cancer risk prediction.

### Definitions

Unless defined otherwise, all technical and scientific terms used herein generally have the same meaning as commonly understood by one of ordinary skill in the art to which this technology belongs. As used in this specification and the appended claims, the singular forms “a,” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, “fibroglandular tissue” means a mixture of fibrous connective tissue (the stroma) and the functional (or glandular) epithelial cells that line the ducts of the breast (the parenchyma) of a subject. “Fibroglandular density” compares the amount of fibrous connective tissue and glandular tissue to the amount of fatty tissue. BI-RADS groups breast density into four different categories:
Fatty breast tissue. Fatty breast tissue is when the breasts are composed almost entirely of non-dense fatty tissue.
Scattered fibroglandular breast tissue. This category includes breasts that have scattered areas of dense tissue but have a higher ratio of non-dense fat.
Heterogeneously dense breast tissue. For this category, the breast includes some non-dense fat, but much of the tissue in the breast is fibroglandular.
Extremely dense breast tissue. When most of the tissue in the breast is dense, the density is considered “extreme.”

In addition to making breast cancer harder to detect using a mammogram, dense breasts are an independent risk factor for breast cancer. The chance of breast cancer tends to increase with breast density.

As used herein, the terms “individual,” “patient,” or “subject” can mean an individual organism, a vertebrate, a mammal, or a human. In some embodiments, the individual, patient or subject is a human.

### Existing Methods for Quantitatively Measuring Mammographic Density

Several approaches for quantitative measurement of mammographic density have been developed for research and clinical practice. The most commonly used research method is Cumulus, a semi-automated thresholding software in which a trained user identifies the intensity level that divides the breast into ‘dense’ and ‘non-dense’ compartments, outputting mammographic dense area (MDA) and percent density (5). While CUMULUS has good reproducibility (6), the approach is not objective, and the selection of dense areas based on intensity thresholds alone is an oversimplification that introduces statistical noise when applied to risk prediction. A number of machine learning approaches have been applied to automate mammographic density, including support vector machines, fuzzy c-means clustering and neural networks (7-12). Proprietary clinical software, such as Volpara and DenSeeMammo, produce fully automated estimates of percent density and were developed based on qualitative BI-RADS density assessments (6). The majority of these methods rely on hand-engineered features selected by experienced radiologists and have varying degrees of accuracy and clinical validation.

Convolutional neural networks (CNNs) are a class of deep learning models that are well-suited to image analysis problems and have recently been applied to mammographic density estimation (9,11,12). CNNs include multiple layers of learnable convolution and pooling filters, where the output of each layer serves as the input for the next layer (13). The majority of the CNN based approaches for mammograms perform classification into qualitative BI-RADS categories rather than segmentation of the dense regions (9,10,14). Existing approaches that are trained to segment dense areas are either not optimized end-to-end (15) or work patch-wise as opposed to training on the whole image (16), increasing complexity and reducing generalizability to other sources of mammograms. Therefore, there is a need for methods that are (a) trained on quantitative segmentation masks as ground truth; (b) are optimized end-to-end; and (c) use the whole breast as input.

### Systems and Methods of Automatically Determining Density of Fibroglandular Tissue (FGT) from Mammograms

The present disclosure relates generally to methods and systems for automated determination of the density or amount of fibroglandular tissue (FGT) from mammograms obtained from subjects, and the application of machine learning to categorize samples based on mammographic density to assess the risk for breast cancer.

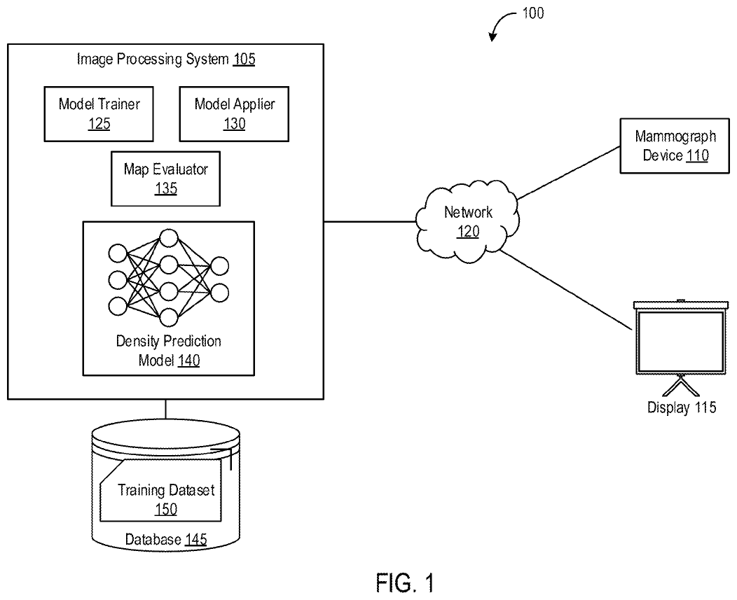

Referring now to FIG. 1, depicted is a block diagram of a system 100 for determining density values of breasts from mammograms. In overview, the system 100 may include at least one image processing system 105, at least one mammograph device 110, and at least one display 115, communicatively coupled via at least one network 120. The image processing system 105 may include at least one model trainer 125, at least one model applier 130, at least one map evaluator 135, at least one density prediction model 140, and at least one database 145, among others. The database 145 may include at least one training dataset 150. Each of the components in the system 100 as detailed herein may be implemented using hardware (e.g., one or more processors coupled with memory) or a combination of hardware and software as detailed herein in Example 4. Each of the components in the system 100 may implement or execute the functionalities detailed herein, such as those described in Examples 1 and 2.

In further detail, the image processing system 105 may (sometimes herein generally referred to as a computing system or a server) be any computing device comprising one or more processors coupled with memory and software and capable of performing the various processes and tasks described herein. The image processing system 105 may be in communication with the network 120. The image processing system 105 may be situated, located, or otherwise associated with at least one server group. The server group may correspond to a data center, a branch office, or a site at which one or more servers corresponding to the image processing system 105 is situated.

Within the image processing system 105, the model trainer 125 may initialize, train, and establish the density prediction model 140 using the training dataset 150. The model applier 130 may apply mammograms to the density prediction model 140 to generate segmentation maps. The map evaluator 135 may use the segmentation maps outputted by the density prediction model 140 to generate information.

The image processing system 105 itself and the components therein, such as the model trainer 125, the model applier 130, the map evaluator 135, and the density prediction model 140 may have a training mode and a runtime mode (sometimes herein referred to as an evaluation or inference mode). Under the training mode, the image processing system 105 may invoke the model trainer 125 to train the density prediction model 140 using the training dataset 150 (e.g., in accordance with supervised learning techniques). Under the runtime, the image processing system 105 may invoke the model applier 130 to apply the density prediction model 140 to acquired images from the mammograph device 110.

The mammograph device 110 (sometimes herein generally referred to as an imaging device or an image acquirer) may be any device for acquiring mammograms of breast regions of subjects. The subjects may be at risk of or may have been diagnosed with breast cancer. The subjects may be under guidance of clinician or hospital staff while scanned by the mammograph device 110. The mammograph device 110 may perform the scan in accordance with any number of imaging modalities, such as X-ray scan, a computed tomography (CT) scan, a computed tomography laser mammography (CTLM), a magnetic resonance imaging (MRI) scan, a nuclear magnetic resonance (NMR) scan, an ultrasound imaging scan, a positron emission tomography (PET) scan, or a photoacoustic spectroscopy scan, among others.

The display 115 may be communicatively coupled with the image processing system 105 or any other computing device comprising one or more processors coupled with memory and software and capable of performing the various processes and tasks described herein. The display 115 may display, render, or otherwise present any information provided by the image processing system 105 or the mammograms of subjects acquired via the mammograph device 110. The information may be used by a clinician examining a subject in diagnosing and deciding which treatment (e.g., a radiation therapy, immunotherapy, chemotherapy, or surgery) to administer to the subject.

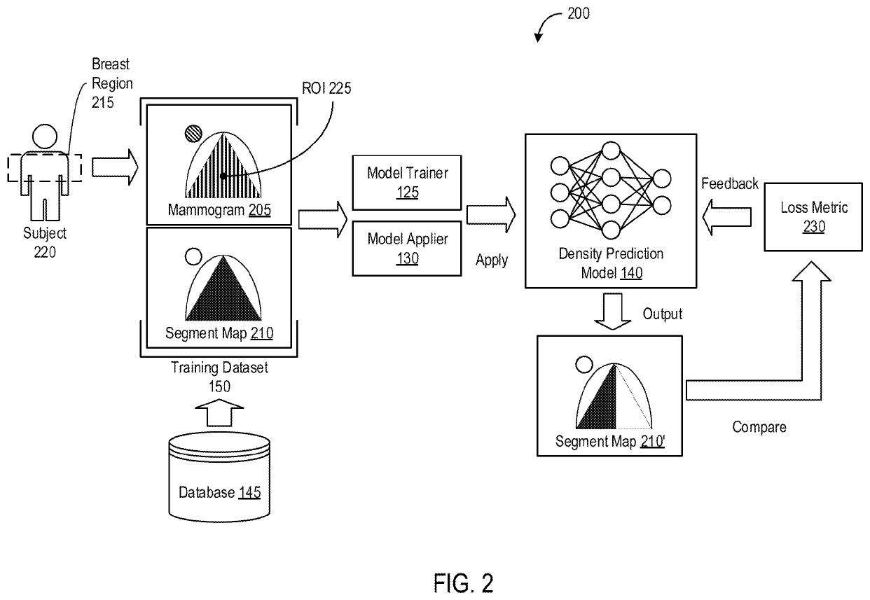

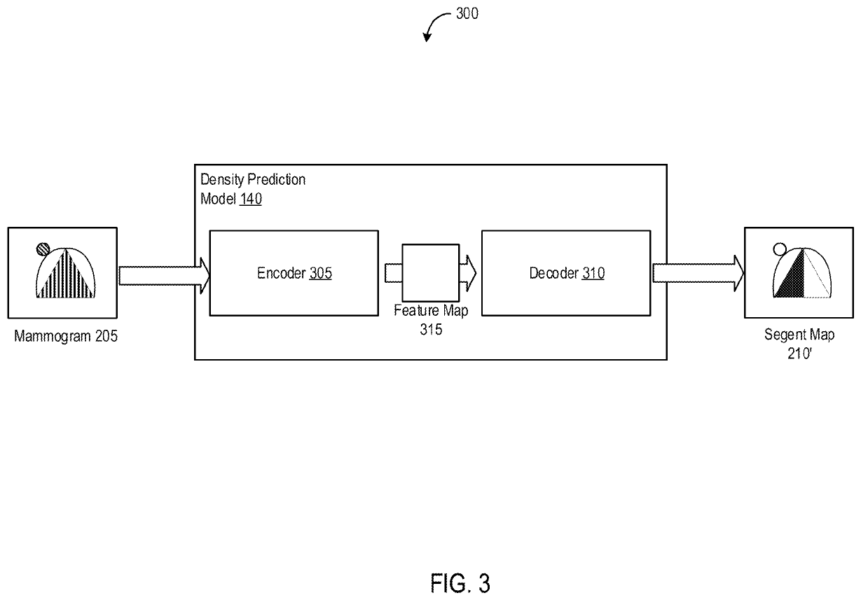

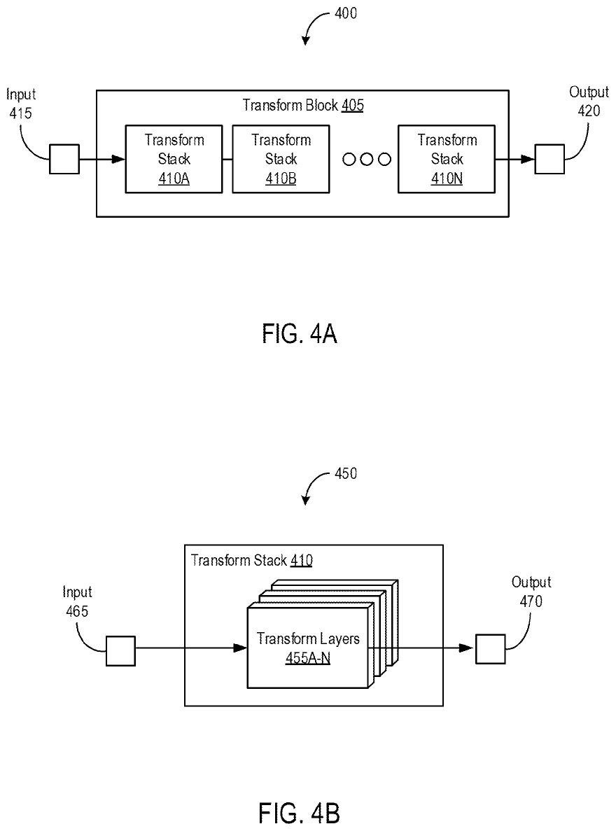

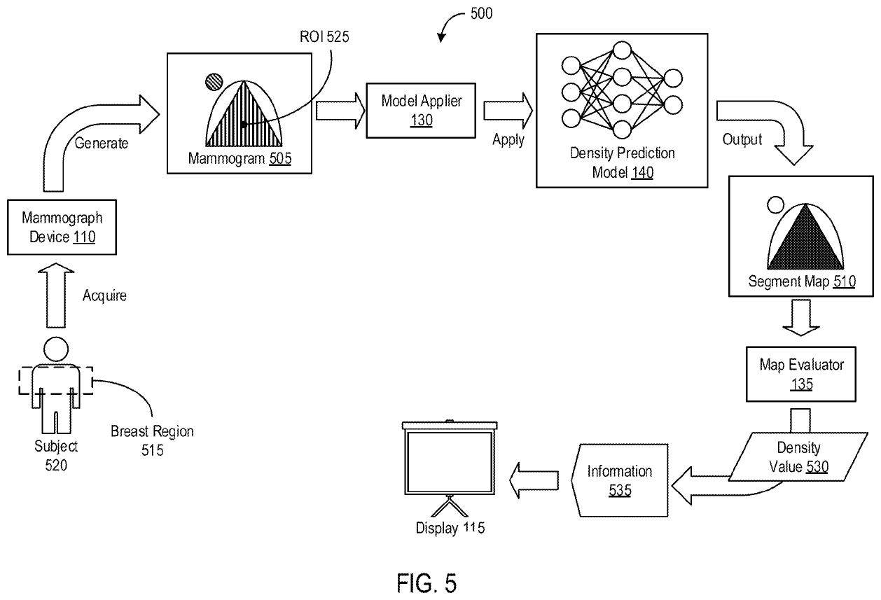

Referring now to FIG. 2, depicted is a block diagram of a process 200 for training a density prediction model in a system 100 for determining density values of breasts from mammograms. The process 200 may include or correspond to operations performed in the system 100 for training the density prediction model 140. Under the process 200, the model trainer 125 executing on the image processing system 105 may initialize and establish the density prediction model 140. The density prediction model 140 may have a set of weights (sometimes herein referred to as kernel parameters, kernel weights, or parameters) to generate segmentation maps from biomedical images (e.g., mammograms). The set of weights may be arranged in a set of transform layers with one or more connections with one another to relate inputs and outputs of the density prediction model 140. In some embodiments, the density prediction model 140 may be implemented using the U-net algorithm as described herein in Examples 1 and 2. In some embodiments, the density prediction model 140 may be implemented using the architecture as detailed herein in conjunction with FIGS. 3-4B. In initializing, the model trainer 125 may calculate, determine, or otherwise generate the initial values to assign the set of weights of the density prediction model 140 using pseudo-random values or fixed defined values.

The model trainer 125 may retrieve, receive, or otherwise identify the training dataset 150 to be used to train the density prediction model 140. In some embodiments, the model trainer 125 may access the database 145 to fetch, retrieve, or identify the training dataset 150. The training dataset 150 may identify or include a set of examples. Each example may identify or include at least one mammogram 205 (sometimes herein referred to as a biomedical image, an image, or a tomogram) and at least one segmentation map 210 (sometimes herein referred to as a segmentation mask, mask, or segmented image), among others. In some embodiments, the model trainer 125 may identify or select a subset of examples from the training dataset 150 to use to train the density prediction model 140. For instance, the model trainer 125 may select 60%-80% of the examples from the training dataset 150 to train the density prediction model 140, while setting aside the remainder of examples in the training dataset 150 for validation.

In each example, the mammogram 205 may be derived, acquired, or otherwise be of a breast region 215 of a subject 220 (e.g., a human patient or an animal). The subject 220 may be at risk of developing breast cancer or may be diagnosed with breast cancer. The breast cancer may be, for example, one of the following: HER2-positive breast cancer, estrogen receptor-positive breast cancer, progesterone receptor-positive breast cancer, or triple negative breast cancer, among others. The incidence of breast cancer may be correlated with a density of the fibroglandular tissue in the breasts of the subject 220. The breast region 215 may generally correspond to one or both breasts (or prominences) located on an upper ventral region of a torso of the subject 220. The breast region 215 from which the mammogram 205 is obtained may encompass, contain, or otherwise include at least one of two breasts of the subject 220. The breast region 215 may include or have at least one dense area (region or volume) corresponding to portions of breast with dense tissue (e.g., scattered fibroglandular density, heterogeneously dense, or extremely dense tissue). The mammogram 205 may be obtained before diagnosis of the breast cancer, prior to a treatment of the breast cancer in the subject 220, or subsequent to such treatment of the breast cancer, among others.

The mammogram 205 may be acquired using any number of imaging modalities in accordance with mammography techniques. For example, the mammogram 205 may include an X-ray scan, a computed tomography (CT) scan, a computed tomography laser mammography (CTLM), a magnetic resonance imaging (MRI) scan, a nuclear magnetic resonance (NMR) scan, an ultrasound imaging scan, a positron emission tomography (PET) scan, or a photoacoustic spectroscopy scan, among others, of the breast region 215 of the subject 220. The mammogram 205 may include a set of two-dimensional cross-sections (e.g., a front, a sagittal, a transverse, or an oblique plane) acquired from the three-dimensional volume. The mammogram 205 may be defined in terms of pixels, in two-dimensions or three-dimensions. Although primarily discussed in terms of X-rays, other imaging modalities besides those listed above may be supported by the image processing system 105 for the mammogram 205. The mammogram 205 may be in the form of an image file (e.g., with a BMP, TIFF, LJPEG, or PNG, among others).

The mammogram 205 may identify or have at least one region of interest (ROI) 225 (also referred herein as a structure of interest (SOI), a volume of interest (VOI), or feature of interest (FOI)). The ROI 225 may correspond to an area, a section, or a portion of the mammogram 205 correlated or associated with the one or more dense areas in the breast region 215 of the subject 220. For instance, the ROI 225 may be a portion of the mammogram 205 corresponding to portions of the breast with dense tissue (e.g., scattered fibroglandular density, heterogeneously dense, or extremely dense tissue) within the subject 220. The portion of the mammogram 205 outside the ROI 225 may correspond to portions of the breast that are non-dense (e.g., fatty breast tissue) in the subject 220. The ROI 225 may correspond to a contiguous portion (e.g., as depicted) or one or more non-contiguous portions within the mammogram 205.

In addition, the segmentation map 210 may at least partially define or identify the at least one ROI 225 in the mammogram 205 of the respective example in the training dataset 150. The segmentation map 210 may identify portions (e.g., pixel locations) within the mammogram 205 that correspond to or are correlated with the dense area of the breast region 215 of the subject 220. For example, the segmentation map 210 may be a matrix corresponding to the dimensions of the mammogram 205, with a value of “1” for pixel locations associated with the ROI 225 and a value of “0” for pixel locations outside the ROI 225. The segmentation map 210 may have been manually inputted, created, or otherwise generated by a clinician examining the mammogram 205 for the set of conditions. For example, the clinician may use a graphical user interface to draw or indicate pixels in the mammogram 205 corresponding to the ROI 225 to form the segmentation map 210. The segmentation map 210 may be in the form of an image file (e.g., with a BMP, TIFF, LJPEG, or PNG, among others).

In some embodiments, the model trainer 125 may create or generate the segmentation map 210 for the mammogram 205 in the example of the training dataset 150. The generation of the segmentation map 210 may be in accordance with a thresholding function applied to the mammogram 205. The portion of the mammogram 205 corresponding to the ROI 225 may have visual characteristics indicative of the dense tissue area in the breast region 215 of the subject 220 that differ from the portion of the mammogram 205 outside the ROI 225. The thresholding function may include, for example, histogram-based thresholding, clustering-based thresholding, entropy-based thresholding, or spatial methods, among others. The thresholding function may be, for example, part of a computer-assisted, semi-automated thresholding method provided by CUMULUS software to differentiate the portion of the mammogram 205 corresponding to the ROI 225 from the portion of the mammogram 205 outside the ROI 225. A clinician examining the mammogram 205 may use the thresholding function of the program applied to the mammogram 205 to generate the segmentation map 210.

With the identification of the example, the model applier 130 executing on the image processing system 105 may feed or apply the mammogram 205 of each example in the training dataset 150 to the density prediction model 140. In feeding, the model applier 130 may process the mammogram 205 in accordance with the set of weights of the density prediction model 140. From processing using the weights of the density prediction model 140, the model applier 130 may produce, create, or otherwise generate a segmentation map 210′. The segmentation map 210′ may be similar to the segmentation map 210 and may at least partially define or identify the at least one ROI 225 in the mammogram 205. The segmentation map 210′ may identify portions (e.g., pixel locations) within the mammogram 205 that correspond to or are correlated with the dense area of the breast region 215 of the subject 220. The segmentation map 210′ may be used to calculate, generate, or otherwise determine a density value characterizing the dense area of the breast region 215 of the subject 220. The model applier 130 may traverse through the selected examples in the training dataset 150 and apply the mammogram 205 from each example to the density prediction model 140.

In some embodiments, the model applier 130 may carry out, execute, or otherwise perform one or more pre-processing functions on the mammogram 205, prior to application to the density prediction model 140. In some embodiments, the model applier 130 may re-size the dimensions of the mammogram 205 to dimensions compatible with the input of the density prediction model 140. The re-sizing may include, for example, bicubic interpolation to alter dimensions and median filtering to reduce noise within the mammogram 205. In some embodiments, the model applier 130 may also perform edge detection (e.g., Canny, Deriche, differential, Sobel, Prewett, and Roberts cross edge detection) on the mammogram 205 to detect, recognize, or otherwise identify an outline of the breasts within the mammogram 205. In some embodiments, the model applier 130 may apply thresholding (e.g., Otsu's thresholding) to remove an outer epidermis layer from the breast area within the mammogram 205. With the application of the one or more pre-processing techniques, the model applier 130 may feed the mammogram 205 to the density prediction model 140 to output the segmentation map 210′.

With the generation, the model trainer 125 may calculate, generate, or otherwise determine at least one loss metric 230 to be used to modify, adjust, or otherwise update the weights of the density prediction model 140. The loss metric 230 may correspond to a degree of deviation between the expected segmentation map 210 as identified in the example of the training dataset 150 and the generated segmentation map 210′ as outputted by the density prediction model 140. To determine, the model trainer 125 may compare the segmentation map 210′ with the segmentation map 210 associated with the mammogram 205 that was fed into the density prediction model 140. The comparison may be a pixel-by-pixel comparison between the segmentation map 210 and the segmentation map 210′. From comparing, the model trainer 125 may determine the loss metric 230 to indicate the degree of deviation. The loss metric 230 may be generated using any number of loss functions, such as a norm loss (e.g., L1 or L2), mean absolute error (MAE), mean squared error (MSE), a quadratic loss, a cross-entropy loss, and a Huber loss, among others. In some embodiments, the model trainer 125 may determine a similarity metric as a function of the segmentation map 210 and the segmentation map 210′. The similarity metric may be used as the loss metric 230, and the function used to calculate the similarity metric may include, for example, a Dice co-efficient metric, a Jaccard index, or an overlap coefficient, among others.

Using the loss metric 230, the model trainer 125 may modify, change, or otherwise update at least one weights of the density prediction model 140. The updating of the weights may be in accordance with backpropagation algorithm and may include dropping units and connections within the density prediction model 140. From updating, the model trainer 125 may encode the density prediction model 140 to generate segmentation maps 210′ to accurately and precisely identify ROIs 225 within the mammogram 205 and by extension determine density values for characterizing the dense area within the breast region 215 of the subject 220. The updating of weights of the density prediction model 140 may be in accordance with an optimization function (also referred herein as an objective function). The optimization function may define one or more rates or parameters at which the weights of the density prediction model 140 are to be updated. The optimization function may be in accordance with stochastic gradient descent, and may include, for example, an adaptive moment estimation (Adam), implicit update (ISGD), and adaptive gradient algorithm (AdaGrad), among others. The updating of the weights of the density prediction model 140 may be repeated until convergence. Upon completion of training, the model trainer 125 may store and maintain the set of weights of the density prediction model 140 on the database 145 to be used to generate segmentation maps from newly acquired mammograms.

Referring now to FIG. 3, depicted is a block diagram of an architecture 300 for the density prediction model 140 in the system 100 for determining density values of breasts from mammograms. Under the architecture 300, the density prediction model 140 may include at least one encoder 305 and at least one decoder 310, among others. The set of weights of the density prediction model 140 may be configured, arrayed, or otherwise arranged across the encoder 305 and the decoder 310 of the density prediction model 140. The inputs and outputs of encoder 305 and the decoder 310 may be connected in any configuration, such as in series (e.g., as depicted), in parallel, or any combination thereof. The architecture of the encoder 305 and the decoder 310 for the architecture 300 is detailed herein below in conjunction with FIGS. 4A and 4B.

The density prediction model 140 may have at least one input and at least one output. The input and output may be related to one another via the set of weights arranged across the encoder 305 and the decoder 310. The input for the density prediction model 140 may include at least one mammogram 205 and may correspond to an input of the encoder 305. In the density prediction model 140, the encoder 305 may generate at least one feature map 315 using the input mammogram 205. The feature map 315 may be a lower dimensional representation of the corresponding input mammogram 205 in a latent feature space. The output of the encoder 305 may be fed forward to the decoder 310. Using the feature map 315 from the encoder 305, the decoder 310 in turn may generate the segmentation map 210′. The segmentation map 210′ may correspond to output for the density prediction model 140 and may identify the ROI 225 within the input mammogram 205.

Referring now to FIG. 4A, depicted is a block diagram of an architecture 400 of a transform block 405 in the density prediction model 140 of the system 100 for determining density values of breasts. The transform block 405 may be used to implement the encoder 305 and the decoder 310 in the density prediction model 140. For example, the encoder 305 and the decoder 310 may each be an instance of the transform block 405.

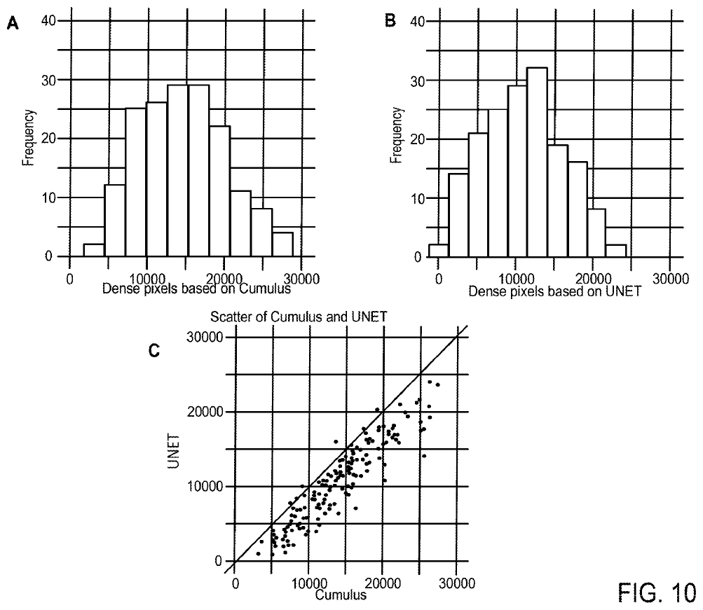

Under the architecture 400, the transform block 405 may include one or more transform stacks 410A-N (hereinafter generally referred to as a transform stack 410). The set of transform stacks 410 can be arranged in series (e.g., as depicted) or parallel configuration, or in any combination. The set of transform stacks 410, for example, may be arranged as shown in FIG. 9 in accordance with the U-net like architecture. In a series configuration, the input of one transform stack 410 may include the output of the previous transform stack 410 (e.g., as depicted). In parallel configuration, the input of one transform stack 410 may include the input of the entire transform block 405.

The transform block 405 may include at least one input 415 and at least one output 420. The set of weights of the encoder 305 or the decoder 310 may be arranged across the transform stacks 410 may define the relationship between the input 415 and the output 420. When used to implement the encoder 305, the input 415 may correspond to the mammogram 205, and the output 420 may be the feature map 315. When used to implement the decoder 310, the input 415 may include the feature map 315 generated by the encoder 305, and the output 420 may be the segmentation map 210′ for the overall density prediction model 140. The size of the transform stacks 410 may vary throughout the transform block 405. For example, the transform stacks 410 of the encoder 305 may decrease from 224×224 to 14×14. Conversely, the transform stacks 410 of the decoder 310 may increase from 256×1 to 224×224.

Referring now to FIG. 4B, depicted is a block diagram of an architecture 450 of a set of transform layers in the transform stack 410 in the density prediction model 140 of the system 100 for determining density values of breasts. The transform stack 410 may be used to implement the encoder 305 and the decoder 310. Under the architecture 450, the transform stack 410 may include a set of transform layers 455A-N (hereinafter generally referred to as transform layers 455). The transform stack 410 may include at least one input 465 and at least one output 470. The input 465 and the output 470 may be related to each other via the set of kernel parameters defined across the transform layers 455. The set of transform layers 455 can be arranged in any configuration such as in series or in parallel, or any combination thereof. For example, under series configuration, the transform layers 455 may have an output of one transform layer 455 fed as an input to a succeeding transform layer 455.

Each transform layer 455 may have a non-linear input-to-output characteristic. The transform layer 455 may comprise a convolutional layer, a normalization layer, and an activation layer (e.g., a rectified linear unit (ReLU)), among others. When used to implement the encoder 305, the transform layers 455 of the transform stack 410 may be configured or arranged as a convolutional neural network (CNN) or a transformer neural network. For example, the convolutional layer, the normalization layer, and the activation layer (e.g., a softmax function, sigmoid non-linearity function, or rectified linear unit (ReLU)) in the transform layers 455 may be arranged in accordance with a fully convolutional neural network (FCNN). When used to implement the encoder 305, the transform layer 455 may include at least one pooling or down-sampling operator layer. When used to implement the decoder 310, the transform layer 455 may include at least one up-sampling operator layer.

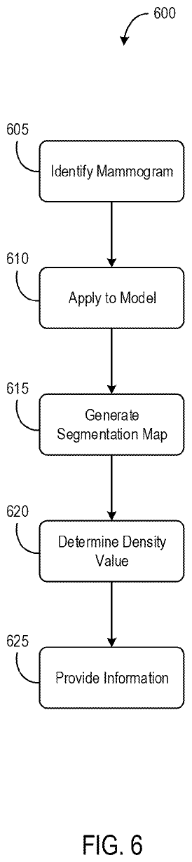

Referring now to FIG. 5, depicted is a block diagram of a process 500 for applying the density prediction model 140 to acquired mammograms in a system 100 for determining density values of breasts from mammograms. The process 500 may include or correspond to operations performed in the system 100 for applying the density prediction model 140 to newly acquired mammograms. The operations of the process 500 may be similar to one or more of the operations in the process 200 as discussed above. Under the process 500, the mammograph device 110 may output, produce, or otherwise generate at least one mammogram 505 of a breast region 515 of a subject 520.

The mammogram 505 may be scanned, obtained, or otherwise of the breast region 515 of the subject 520. The mammogram 505 may be similar to the mammogram 205 detailed herein above. The subject 520 may be at risk of developing breast cancer or may be diagnosed with breast cancer. The breast cancer may be, for example, one of the following: HER2-positive breast cancer, estrogen receptor-positive breast cancer, progesterone receptor-positive breast cancer, or triple negative breast cancer, among others. The incidence of breast cancer may be correlated with a density of the fibroglandular tissue in the breasts of the subject 520. The breast region 515 may generally correspond to one or both breasts (or prominences) located on an upper ventral region of a torso of the subject 520. The breast region 515 from which the mammogram 505 is obtained may encompass, contain, or otherwise include at least one of two breasts of the subject 520. The breast region 515 may include or have at least one dense area (region or volume) corresponding to portions of breast with dense tissue (e.g., scattered fibroglandular density, heterogeneously dense, or extremely dense tissue). The mammogram 505 may be obtained via the mammograph device 110 before diagnosis of the breast cancer, prior to a treatment of the breast cancer in the subject 520, or subsequent to such treatment of the breast cancer, among others.

The mammogram 505 may be acquired using any number of imaging modalities in accordance with mammography techniques. For example, the mammogram 505 may include an X-ray scan, a computed tomography (CT) scan, a computed tomography laser mammography (CTLM), a magnetic resonance imaging (MRI) scan, a nuclear magnetic resonance (NMR) scan, an ultrasound imaging scan, a positron emission tomography (PET) scan, or a photoacoustic spectroscopy scan, among others, of the breast region 515 of the subject 520. The mammogram 505 may include a set of two-dimensional cross-sections (e.g., a front, a sagittal, a transverse, or an oblique plane) acquired from the three-dimensional volume. The mammogram 505 may be defined in terms of pixels, in two-dimensions or three-dimensions. Although primarily discussed in terms of X-rays, other imaging modalities besides those listed above may be supported by the image processing system 105 for the mammogram 505.

The mammogram 505 may identify or have at least one region of interest (ROI) 525 (also referred herein as a structure of interest (SOI), a volume of interest (VOI), or feature of interest (FOI)). The ROI 525 may correspond to an area, a section, or a portion of the mammogram 505 correlated or associated with the one or more dense areas in the breast region 515 of the subject 520. For instance, the ROI 525 may be a portion of the mammogram 505 corresponding to portions of the breast with dense tissue (e.g., scattered fibroglandular density, heterogeneously dense, or extremely dense tissue) within the subject 520. The portion of the mammogram 505 outside the ROI 525 may correspond to portions of the breast that are non-dense (e.g., fatty breast tissue) in the subject 520. The ROI 525 may correspond to a contiguous portion (e.g., as depicted) or one or more non-contiguous portions within the mammogram 505.

Upon acquisition, the mammograph device 110 (or a computing device coupled thereto) may provide, send, or otherwise transmit the mammogram 505 to the image processing system 105. The mammogram 505 may be provided with metadata including, for example, an anonymized identifier for the subject 520, a timestamp of acquisition of the mammogram 505, and device manufacturer information for the mammograph device 110, among others. In some embodiment, the mammograph device 110 may send the mammogram 505 for storage and maintenance on a database (e.g., the database 145) for subsequent processing by the image processing system 105. In some embodiments, the mammograph device 110 may provide the mammogram 505 to the image processing system 105 in response to a request to process the mammogram 505. The request may be inputted by a clinician examining the subject 520 to diagnose and treat for breast cancer.

The model applier 130 may retrieve, receive, or otherwise identify the mammogram 505 from the mammograph device 110. With the identification, the model applier 130 may feed or apply the mammogram 505 to the density prediction model 140. The density prediction model 140 may have been established as discussed above under process 200. In feeding, the model applier 130 may process the mammogram 505 in accordance with the set of weights of the density prediction model 140. From processing using the weights of the density prediction model 140, the model applier 130 may produce, create, or otherwise generate a segmentation map 510. The segmentation map 510 may at least partially define or identify the at least one ROI 525 in the mammogram 505. The segmentation map 510 may identify portions (e.g., pixel locations) within the mammogram 505 that correspond to or are correlated with the dense area of the breast region 515 of the subject 520. The segmentation map 510 may be used to calculate, generate, or otherwise determine a density value characterizing the dense area of the breast region 515 of the subject 520.

In some embodiments, the model applier 130 may carry out, execute, or otherwise perform one or more pre-processing functions on the mammogram 505, prior to application to the density prediction model 140. In some embodiments, the model applier 130 may re-size the dimensions of the mammogram 505 to dimensions compatible with the input of the density prediction model 140. The re-sizing may include, for example, bicubic interpolation to alter dimensions and median filtering to reduce noise within the mammogram 505. In some embodiments, the model applier 130 may also perform edge detection (e.g., Canny, Deriche, differential, Sobel, Prewett, and Roberts cross edge detection) on the mammogram 505 to detect, recognize, or otherwise identify an outline of the breasts within the mammogram 505. In some embodiments, the model applier 130 may apply thresholding (e.g., Otsu's thresholding) to remove an outer epidermis layer from the breast area within the mammogram 505. With the application of the one or more pre-processing techniques, the model applier 130 may feed the mammogram 505 to the density prediction model 140 to output the segmentation map 510.

Based on the segmentation map 510, the map evaluator 135 executing on the image processing system 105 may calculate, generate, or otherwise determine at least one density value 530. The density value 530 may characterize the dense tissue area of the breast region 515 of the subject 520. The dense area of the breast region 515 may correspond to the ROI 525 of the mammogram 505, and in turn may be highlighted, labeled, or otherwise identified by the segmentation map 510. The segmentation map 510 may identify or have at least one portion corresponding to the ROI 525 and at least one portion outside the ROI 525 of the associated mammogram 505. For instance, a matrix forming the segmentation map 510 may have a set of elements set to “1” for pixel locations corresponding to the ROI 525 and a set of elements set to “0” for pixel locations corresponding to portions outside the ROI 525.

To determine, the map evaluator 135 may calculate, determine, or otherwise identify a ratio (or fraction or percentage) between the portion of the segmentation map 510 identifying the ROI 525 and the portion of the segmentation map 510 outside the ROI 525. The ratio may be, for example, between a number of pixels for the portion of the segmentation map 510 identifying the ROI 525 and a number of pixels for the portion of the segmentation map 510 outside the ROI 525. With the determination, the map evaluator 135 may store and maintain an association between the subject 520 (e.g., using an identifier) and the density value 530, using one or more data structures (e.g., arrays, matrixes, tables, linked lists, stacks, queues, trees, or heaps). The association may be among the subject 520, the density value 530, and the mammogram 505.

In some embodiments, the map evaluator 135 may identify or select the density value 530 from one of the following groups (e.g., as defined under Breast Imaging-Reporting and Data System (BI-RAD)) from least dense to more dense: a fatty breast tissue, scattered fibroglandular breast tissue, heterogeneously dense breast tissue, or extremely dense breast tissue, as described herein above. The density value 530 may define, identify, or may be a measure of density of fibroglandular tissue in the breast region 515 of the subject 520. The selection may be based on the determined density value 530 characterizing the dense area within the breast region 515 of the subject 520. In general, the higher the density value 530, the more likely the density value 530 may be selected as heterogeneously dense breast tissue or extremely dense breast. Conversely, the lower the density value 530, the less likely the density value 530 for the breast region 515 may be identified as a fatty breast tissue or scattered fibroglandular breast tissue.

With the determination, the map evaluator 135 may group, assign, or otherwise classify the subject 520 in one of a set of risk levels based on the density value 530 for the breast region 515 of the subject 520. Each risk level may correspond to or be associated with a likelihood of occurrence of breast cancer in the subject 520. For instance, the risk levels may include “low-risk,” “medium risk,” and “high risk” groups for the subjects 520 at potential risk of breast cancer. Each risk level may be defined or associated with a range of density values 530. If the density value 530 for the subject 520 falls into the range, the map evaluator 135 may assign the subject 520 into the corresponding risk level. The map evaluator 135 may store and maintain the association between the subject 520 and the assigned risk level.

In some embodiments, the map evaluator 135 may group, classify, or otherwise categorize the dense area of the breast region 515 of the subject 520 into one of a set of density types based on the density value 530. The set of density types may include the following, for example, from least dense to more dense: a fatty breast tissue, scattered fibroglandular breast tissue, heterogeneously dense breast tissue, or extremely dense breast tissue. Each density type may be defined or associated with a range of density values. If the density value 530 for the subject 520 falls into the range, the map evaluator 135 may assign the dense area in the breast region 515 of the subject 520 into the corresponding density value. The map evaluator 135 may store and maintain the association between the subject 520 and the assigned density type for the dense area of the breast region 515 of the subject 520.

In some embodiments, the map evaluator 135 may identify or determine whether the subject 520 is to be administered with a treatment for breast cancer based on the density value 530. The treatment may include, for example, a radiation therapy, immunotherapy, chemotherapy or surgery, among others. To determine, the map evaluator 135 may compare the density value 530 for the subject 520 with a threshold. The threshold may define, delineate, or otherwise identify a value for the density value 530 at which the associated subject 520 is to be administered the treatment. The threshold may be determined using the density values measured from a group of control subjects diagnosed as without breast cancer. The threshold, for example, may be set at a value corresponding to fatty breast tissue categorization. If the density value 530 is greater than or equal to the threshold, the map evaluator 135 may determine that the subject 520 is to be administered treatment. Conversely, if the density value 530 is less than the threshold, the map evaluator 135 may determine that the subject 520 is to be not administered treatment. The map evaluator 135 may store and maintain the association between the subject 520 and the determination of whether the subject 520 is to be administered treatment.

Using the association between the subject 520 and the density value 530, the map evaluator 135 may create, determine, or otherwise generate information 535. The information 535 may identify or include any one or more of the following: the identifier for the subject 520, the mammogram 505, the segmentation map 510, and the density value 530 for the subject 520, among others. In some embodiments, the map evaluator 135 may insert or include the determined risk level for the subject 520 or the density type (e.g., fatty breast tissue, scattered fibroglandular breast tissue, heterogeneously dense breast tissue, or extremely dense breast tissue), or both, into the information 535. In some embodiments, the map evaluator 135 may insert or include an indication of the determination of whether the density value 530 is above or less than the threshold into the information 535. In some embodiments, the map evaluator 135 may insert or include an indication of the determination of whether the subject 520 is to be administered with treatment based on the density value 530. With the generation, the map evaluator 135 may send, transmit, or otherwise provide the information 535 to the display 115. The provision of the information 535 may be in response to a request from a user (e.g., clinician examining the subject 520).

The display 115 (or a computing device connected thereto) may display, render, or otherwise present the information 535 from the image processing system 105, such as any one or more of the following: the identifier for the subject 520, the mammogram 505, the segmentation map 510, and the density value 530 for the subject 520, among others. For example, the display 115 may present the information 535 via a graphic user interface of an application to display the mammogram 505, the segmentation map 510, together with the density value 530. In some embodiments, the display 115 may present the determined risk level for the subject 520, the density type, the indication of the determination of whether the density value 530 is above or less than the threshold, or the indication of whether the subject 520 is to be administered with treatment. The information 535 may be presented to the clinician examining the subject 520. The clinician in turn may use the information 535 to diagnose or decide administration of treatment to the subject 520 for breast cancer. For instance, a clinician examining the subject 520 may administer treatment (e.g., radiation therapy, immunotherapy, chemotherapy, or surgery) based on the indication the density value 530 is above the threshold.

In this manner, the image processing system 105 may use machine learning (ML) model techniques to automatically provide more accurate and objective estimations of breast density values using mammograms taken of the breast regions of subjects. This may reduce or eliminate the involvement of manual, tedious, and subjective examination of such mammograms by clinicians, thereby cutting the time expended in providing such assessments. Since the image processing system 105 is able to provide objective metrics for the dense regions in subject's breasts, better diagnosis and care may be provided to such subjects at risk of breast cancer. Furthermore, due to the reduced human involvement in assessing mammograms, the image processing system 105 may be able to lower consumption of computing resources (e.g., processing and memory) as well as network bandwidth that would have been otherwise spent from monitoring and responding to user interaction.

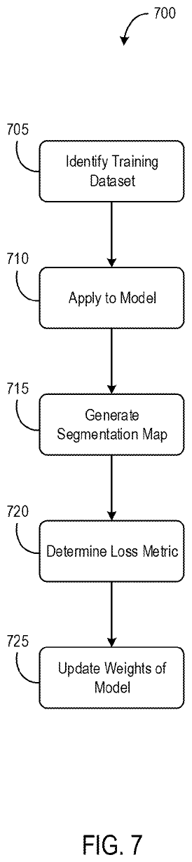

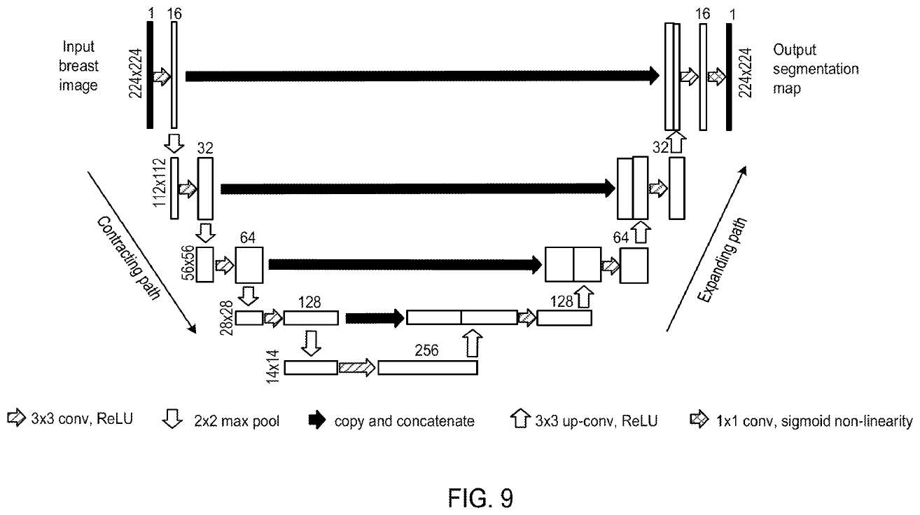

Referring now to FIG. 6, depicted is a flow diagram of a method 600 of determining density values of breasts from mammograms. The method 600 may be implemented using or performed by any of the components described herein, such as the image processing system 105 in conjunction with FIGS. 1-5 or the system 800 in FIG. 8. Under the method 600, a computing system (e.g., the image processing system 105) may identify a mammogram (e.g., the mammogram 505) of a breast region (e.g., the breast region 515) of a subject (e.g., the subject 520) (605). The computing system may apply the mammogram to a model (e.g., the density prediction model 140) (610). The computing system may generate a segmentation map (e.g., the segmentation map 510) from the application of the model (615). The computing system may determine a density value (e.g., the density value 530) using the segmentation map (620). The computing system may provide information (e.g., the information 535) based on the density value (625).

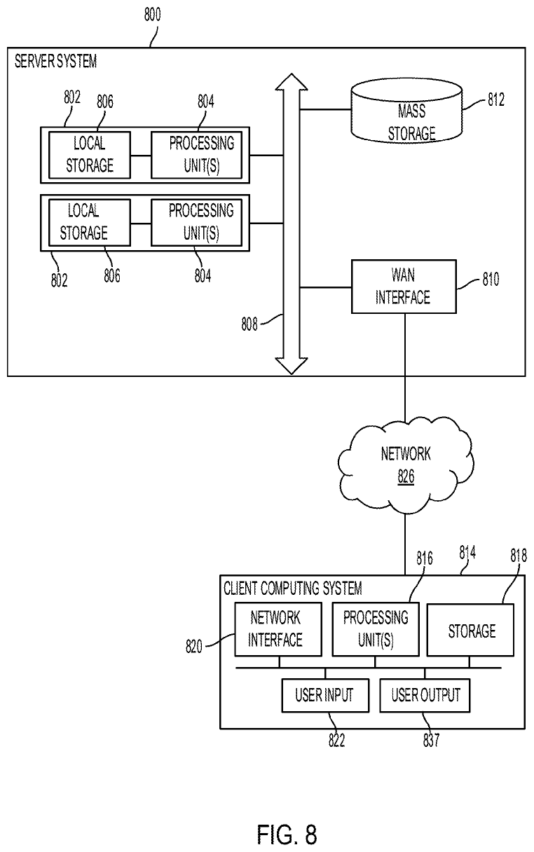

Referring now to FIG. 7, depicted is a flow diagram of a method 700 of training models to determine density values of breasts from mammograms. The method 700 may be implemented using or performed by any of the components described herein, such as the image processing system 105 in conjunction with FIGS. 1-5 or the system 800 in FIG. 8. Under the method 700, a computing system (e.g., the image processing system 105) may identify a training dataset (e.g., the training dataset 150) (705). The computing system may apply a mammogram (e.g., the mammogram 205) of each example in the training dataset to a model (e.g., the density prediction model 140) (710). The computing system may generate a segmentation map (e.g., a segmentation map 210′) from the application of the model (715). The computing system may determine a loss metric (e.g., the loss metric 230) based on a comparison between the output segmentation map and an expected segmentation map (e.g., the segmentation map 210) of the example in the training dataset (720). The computing system may update set of weights of the model using the loss metric (725).

### Network and Computing Environments

Various operations described herein can be implemented on computer systems. FIG. 8 shows a simplified block diagram of a representative server system 800, client computing system 814, and network 826 usable to implement certain embodiments of the present disclosure. In various embodiments, server system 800 or similar systems can implement services or servers described herein or portions thereof. Client computing system 814 or similar systems can implement clients described herein. Server system 800 can have a modular design that incorporates a number of modules 802 (e.g., blades in a blade server embodiment); while two modules 802 are shown, any number can be provided. Each module 802 can include processing unit(s) 804 and local storage 806.

Processing unit(s) 804 can include a single processor, which can have one or more cores, or multiple processors. In some embodiments, processing unit(s) 804 can include a general-purpose primary processor as well as one or more special-purpose co-processors such as graphics processors, digital signal processors, or the like. In some embodiments, some or all processing unit(s) 804 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 804 can execute instructions stored in local storage 806. Any type of processors in any combination can be included in processing unit(s) 804.

Local storage 806 can include volatile storage media (e.g., DRAM, SRAM, SDRAM, or the like) and/or non-volatile storage media (e.g., magnetic or optical disk, flash memory, or the like). Storage media incorporated in local storage 806 can be fixed, removable, or upgradeable as desired. Local storage 806 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 804 need at runtime. The ROM can store static data and instructions that are needed by processing unit(s) 804. The permanent storage device can be a non-volatile read-and-write memory device that can store instructions and data even when module 802 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 806 can store one or more software programs to be executed by processing unit(s) 804, such as an operating system and/or programs implementing various server functions such as functions of the systems 100 or any other system described herein, or any other server(s) associated with systems 100 or any other system described herein.

“Software” refers generally to sequences of instructions that, when executed by processing unit(s) 804, cause server system 800 (or portions thereof) to perform various operations, thus, defining one or more specific machine embodiments that execute and perform the operations of the software programs. The instructions can be stored as firmware residing in read-only memory and/or program code stored in non-volatile storage media that can be read into volatile working memory for execution by processing unit(s) 804. Software can be implemented as a single program or a collection of separate programs or program modules that interact as desired. From local storage 806 (or non-local storage described below), processing unit(s) 804 can retrieve program instructions to execute and data to process in order to execute various operations described above.

In some server systems 800, multiple modules 802 can be interconnected via a bus or other interconnect 808, forming a local area network that supports communication between modules 802 and other components of server system 800. Interconnect 808 can be implemented using various technologies including server racks, hubs, routers, etc.

A wide area network (WAN) interface 810 can provide data communication capability between the local area network (interconnect 808) and the network 826, such as the Internet. Technologies can be used, including wired (e.g., Ethernet, IEEE 802.3 standards) and/or wireless technologies (e.g., Wi-Fi, IEEE 802.11 standards).

In some embodiments, local storage 806 is intended to provide working memory for processing unit(s) 804, providing fast access to programs and/or data to be processed while reducing traffic on interconnect 808. Storage for larger quantities of data can be provided on the local area network by one or more mass storage subsystems 812 that can be connected to interconnect 808. Mass storage subsystem 812 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 812. In some embodiments, additional data storage resources may be accessible via WAN interface 810 (potentially with increased latency).

Server system 800 can operate in response to requests received via WAN interface 810. For example, one of modules 802 can implement a supervisory function and assign discrete tasks to other modules 802 in response to received requests. Work allocation techniques can be used. As requests are processed, results can be returned to the requester via WAN interface 810. Such operation can generally be automated. Further, in some embodiments, WAN interface 810 can connect multiple server systems 800 to each other, providing scalable systems capable of managing high volumes of activity. Other techniques for managing server systems and server farms (collections of server systems that cooperate) can be used, including dynamic resource allocation and reallocation.

Server system 800 can interact with various user-owned or user-operated devices via a wide area network such as the Internet. An example of a user-operated device is shown in FIG. 8 as client computing system 814. Client computing system 814 can be implemented, for example, as a consumer device such as a smartphone, other mobile phone, tablet computer, wearable computing device (e.g., smart watch, eyeglasses), desktop computer, laptop computer, and so on.

For example, client computing system 814 can communicate via WAN interface 810. Client computing system 814 can include computer components such as processing unit(s) 816, storage device 818, network interface 820, user input device 822, and user output device 837. Client computing system 814 can be a computing device implemented in a variety of form factors, such as a desktop computer, laptop computer, tablet computer, smartphone, other mobile computing device, wearable computing device, or the like.

Processing unit(s) 816 and storage device 818 can be similar to processing unit(s) 804 and local storage 806 described above. Suitable devices can be selected based on the demands to be placed on client computing system 814; for example, client computing system 814 can be implemented as a “thin” client with limited processing capability or as a high-powered computing device. Client computing system 814 can be provisioned with program code executable by processing unit(s) 816 to enable various interactions with server system 800.

Network interface 820 can provide a connection to the network 826, such as a wide area network (e.g., the Internet) to which WAN interface 810 of server system 800 is also connected. In various embodiments, network interface 820 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 822 can include any device (or devices) via which a user can provide signals to client computing system 814; client computing system 814 can interpret the signals as indicative of particular user requests or information. In various embodiments, user input device 822 can include any or all of a keyboard, touch pad, touch screen, mouse or other pointing device, scroll wheel, click wheel, dial, button, switch, keypad, microphone, and so on.

User output device 837 can include any device via which client computing system 814 can provide information to a user. For example, user output device 837 can include display-to-display images generated by or delivered to client computing system 814. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 837 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 804 and 816 can provide various functionality for server system 800 and client computing system 814, including any of the functionality described herein as being performed by a server or client, or other functionality.

It will be appreciated that server system 800 and client computing system 814 are illustrative and that variations and modifications are possible. Computer systems used in connection with embodiments of the present disclosure can have other capabilities not specifically described here. Further, while server system 800 and client computing system 814 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

### EXAMPLES

The present technology is further illustrated by the following Examples, which should not be construed as limiting in any way.

### Example 1: Experimental Methods

### Study Population

The source of images for training and testing was the Women's Environment, Cancer, and Radiation Epidemiology (WECARE) Study of contralateral breast cancer (CBC) risk, which included 1,280 images of a single unaffected breast from 649 patients diagnosed with unilateral local/regional breast cancer.

The mammograms used to train and evaluate the algorithm were collected as part of the WECARE (for Women's Environment, Cancer, and Radiation Epidemiology) Study, a case-control study of ≥1-year survivors of breast cancer. The cases included women with contralateral breast cancer (CBC) and the controls were women with unilateral breast cancer (UBC) individually matched to the cases based on age, at-risk time, race/ethnicity, and recruitment site (22). All participants at baseline were diagnosed with a unilateral, local/regional invasive breast cancer and had at least one mammogram of the taken either (a) prior to or at the time of diagnosis of their first breast cancer or (b) after treatment for their first breast cancer. Processed cranio-caudal (CC) film mammograms were centrally digitized at two study sites using a Kodak Lumisys Digital Scanner. MDA was estimated for the unaffected contralateral breast for each mammogram using the semi-automated thresholding software, CUMULUS (5), as previously described (23). There were 1,082 unaffected mammograms from 649 participants (some participants had both pre-and post-treatment mammograms). Only one unaffected breast was assessed for each mammogram. For the algorithm training, case-control status or any other study variables were not taken into account. The ground truth for this study was mammographic dense area (in pixels) measured by CUMULUS (5). Described herein is a fully automated pipeline to estimate MDA using the approach based on the Cumulus MDA ground truth as disclosed herein.

### Mammogram Pre-Processing

Prior to developing the U-net model, each image was pre-processed to extract the breast area. First, each image was resized by bicubic interpolation, and median filtering was applied to reduce image noise. Second, canny edge detection was applied to identify outlines of the breast and any artifacts in the imaging, and morphological image processing was used to separate the breast from non-breast objects. Third, the largest connected object was extracted to identify the breast area. In a scanned film mammogram, the skin layer appears radio-dense, similar to fibroglandular tissue. Therefore, the skin layer was digitally ‘removed’ by segmenting the breast area from the skin using Otsu's threshold selection method (24). As a final pre-processing step, the images were cropped to the breast region and resampled to a common resolution of 224×224 pixels. The resulting breast mask was then used to develop the MDA segmentation algorithm. All image pre-processing was performed in R using the imager (25), EBImage (26), and e 1070 (27) packages.

### Model Development

The images were partitioned at the image level into 70% train, 15% validation and 15% holdout test sets. The training set was used to optimize the weights of the network; the validation set was used for hyperparameter tuning (e.g., determining the number of training epochs using early stopping); and the holdout test set used to measure algorithm generalization performance and evaluate CBC case-control discrimination (see Case-control analysis, below).

The model was a FCNN based on the U-net architecture, consisting of 5 levels of filtering operations in the encoder and the decoder (FIG. 9). Starting with 16 convolutional filters in the first layer, the number of filters were doubled in successive encoding layers and halved in successive decoding layers. The convolutions in the encoding layers and transpose convolutions in the decoding layers were performed using 3×3 kernels, whereas the pooling operations were performed using 2×2 kernels. During each iteration of training, the predicted segmentation map was compared with the ground truth using a differentiable Dice co-efficient metric (28) and was used to train the neural network via backpropagation algorithm (29). Dropout layers were added to both encoding and decoding paths to reduce overfitting by randomly dropping units and their connections from the neural network during training (30), thereby preventing the neurons from excessively co-adapting to the training data (31). The network was trained using the Adam optimizer with the differentiable dice co-efficient as the loss function (28). The validation set was used to identify the optimal number of training iterations using early stopping (29). Early stopping helps to prevent overfitting by identifying the optimal number of training iterations beyond which the performance starts to deteriorate on the validation set (29). The output of the final activation layer was passed through a sigmoid function to convert it to a [0,1] map. The model described herein is referred to as the “U-net”, while noting that the architecture has minor differences from the U-net reported in literature (13).

### Evaluation of Model Performance

Precision-recall (PR) curves [Schütze, Hinrich, Christopher D. Manning, and Prabhakar Raghavan. Introduction to information retrieval. Vol. 39. Cambridge: Cambridge University Press, 2008] as well as Dice coefficient and Jaccard index [Eelbode et al., IEEE Trans Med Imaging. 2020 November; 39(11): 3679-3690] were used to evaluate the performance of the algorithm on the holdout test set. The PR curves were computed in the following way: intensity thresholds varying from 0 to 1 were first applied on the output [0,1] segmentation maps to convert them to binary segmentations (dense/non-dense regions). At each intensity threshold, the precision was computed as the ratio of the overlap between the U-net-predicted MDA (in pixels) and the Cumulus MDA over the total U-net-predicted MDA; and recall was computed as the overlap over the total Cumulus MDA. Lastly, the precision and recall values at each threshold were averaged across all the cases in the test set to compute summary precision and recall at that threshold. The dice coefficient was computed as the ratio of twice the overlap between the predicted MDA and Cumulus MDA over the sum of the predicted and Cumulus MDAs. The Jaccard Index was computed as the ratio of the overlap between the predicted and Cumulus MDAs over the union of the predicted and Cumulus MDAs. In a secondary analysis, the performance of the segmentation was evaluated for an independent set of digital mammograms from the WECARE Study using the model trained on the digitized mammograms.

### Case-Control Analysis

Using the holdout set, a case-control study with 51 CBC cases and 49 UBC controls was completed to compare the association between CBC and MDA measured by the U-Net program (“U-net MDA”) vs. dense area measured by Cumulus (“Cumulus MDA”). Among the cases, 2 had a pre-diagnosis mammogram only; 13 had a post-diagnosis mammogram only; and 36 had both; among the controls, 4 had a pre-diagnosis mammogram only, 13 had a post-diagnosis mammogram only, and 32 had both. For women with both pre-and post-diagnosis mammograms, the median of the two MD measures was used. MD measures were log-transformed. CBC case-control status was regressed on the log-transformed density measures and adjusted for known risk factors: estrogen receptor (ER) status of the first primary breast cancer, age at time of the first breast cancer, chemotherapy and radiotherapy for treatment of the first breast cancer; and matching factors used in the WECARE Study: at-risk time, race/ethnicity, and recruitment registry. Associations between CBC and MD measures were standardized and reported as odds per adjusted standard deviation for the controls, as described previously (32).

### Example 2: Results and Discussion

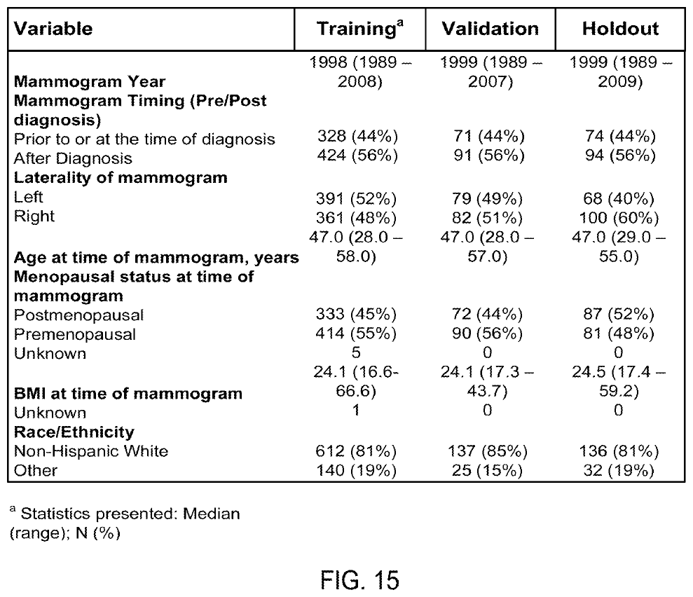

A description of the images in the training, validation, and holdout sets is provided in FIG. 15. The train-validation-test set splits were performed at the patient level. The algorithm was trained using a batch size of 32 for 100 epochs, which was found to be sufficient to determine the optimal number of training iterations using early stopping. The dropout probability was set to 0.05. The Adam optimizer was configured with a learning rate of 0.001, Beta 1 of 0.9, and Beta 2 of 0.999. The learning rate was reduced by a factor of 0.1 if there was no improvement in the loss function for 3 continuous epochs. Tensorflow [doi: zenodo.org/record/5799851] and Keras [Chollet, F., & others. (2015). Keras. GitHub. Retrieved from github.com/fchollet/keras] python libraries were used for implementing and training the models. All computation was performed on an Nvidia Tesla P100 GPU.

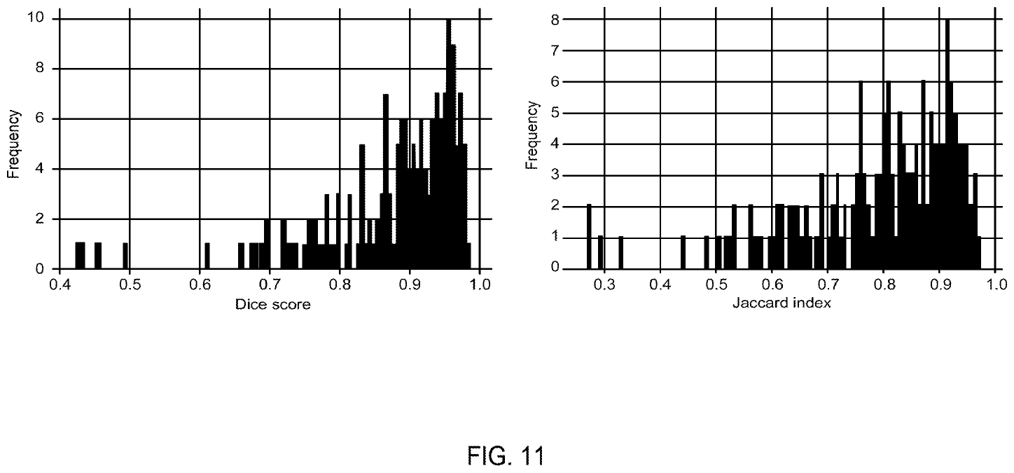

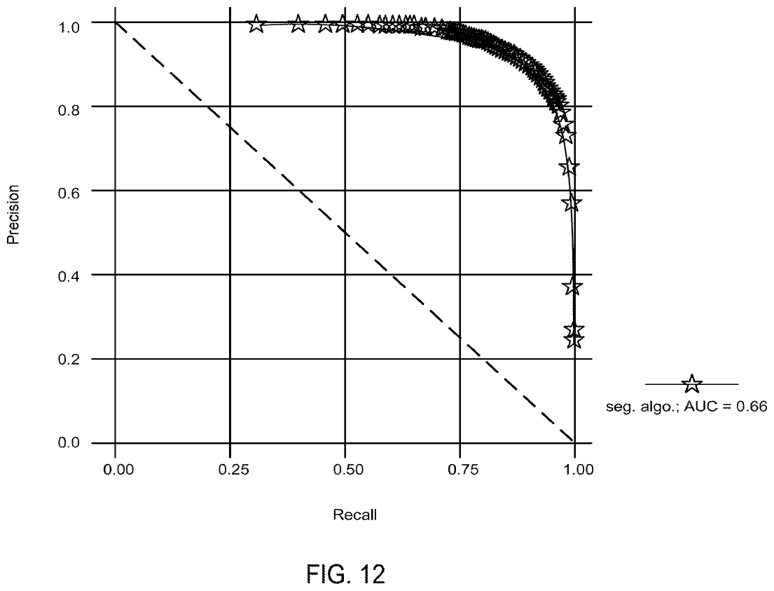

FIG. 10 shows the histograms of the distributions of U-net MDA and Cumulus MDA in the holdout set, as well as a scatter plot comparing the two. The intraclass correlation coefficient for the two measures was 0.92 (95% CI 0.90-0.94). The mean and standard deviation (SD) of the U-net MDA (mean=10,897; SD 5,205) were both lower than that of the Cumulus MDA (mean=14,123; SD=5,481). FIG. 11 shows distributions of dice and Jaccard indices across all test cases. The median dice coefficient for the U-net MDA was 0.91 (range 0.42-0.99; mean=0.87; SD=0.10) and median Jaccard index was 0.83 (range 0.27-0.97; mean=0.79; SD=0.14).

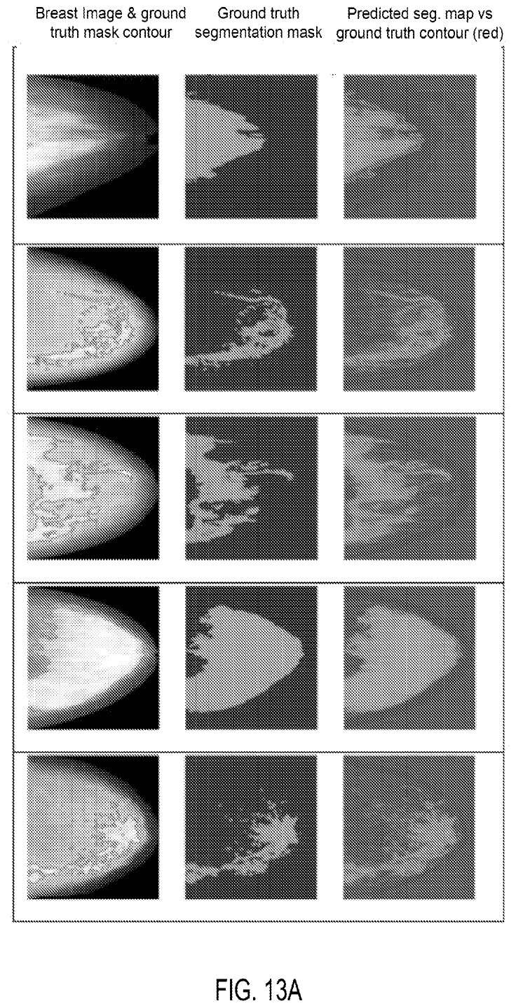

FIG. 12 shows the precision-recall curve computed on the holdout set by varying the intensity thresholds of the predicted segmentation maps. The average precision, computed as the mean of weighted precisions at each threshold [Turpin, Andrew; Scholer, Falk (2006). “User performance versus precision measures for simple search tasks”. Proceedings of the 29th Annual international ACM SIGIR Conference on Research and Development in information Retrieval (Seattle, WA, Aug. 6-11, 2006). New York, NY: ACM: 11-18. doi: 10.1145/1148170.1148176. ISBN 1-59593-369-7; Zhu, Mu. “Recall, precision and average precision.” Department of Statistics and Actuarial Science, University of Waterloo, Waterloo 2, no. 30 (2004): 6)] was 0.98.

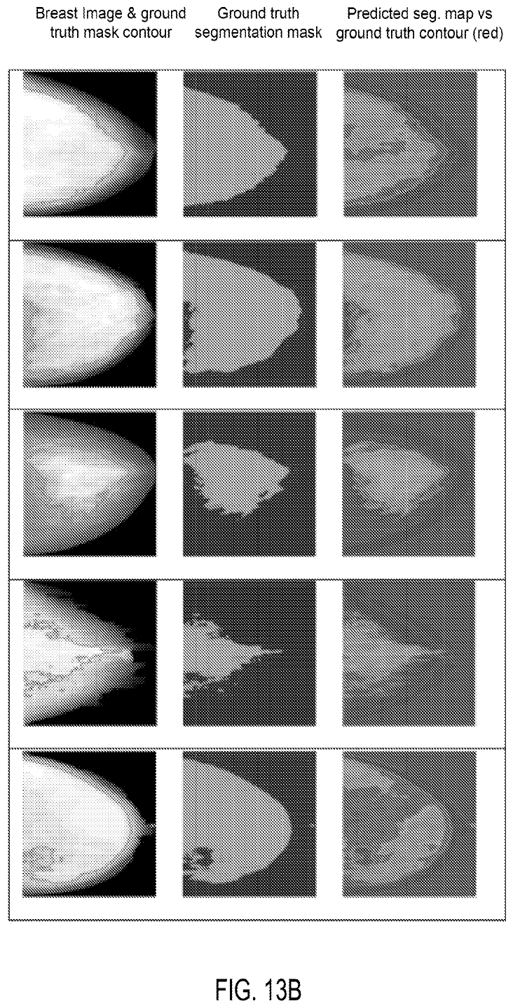

FIGS. 13A and 13B each provides exemplary visualizations of the U-net performance on the holdout set. The breast image and an outline of the CUMULUS determined dense area is shown in (a); the corresponding ground truth segmentation mask is shown in (b); and a heat map of the dense region predicted by the algorithm along with the outline of the CUMULUS ground truth is shown in (c), where higher-temperature regions (more yellow) indicate predicted MDA [The viridis color palettes, cran.r-project.org/web/packages/viridis/830 vignettes/intro-to-viridis.htm]. These results demonstrate the effectiveness of the algorithm even in challenging scenarios where the dense region is split into many irregular areas (see, e.g., rows 2, 3, and 5).

In the secondary analysis of digital images, FIGS. 13A and 13B each provides some examples of the application of the algorithm trained using digitized mammograms on 60 full-field digital mammograms from the WECARE Study. The overall median dice coefficient for the digital mammograms was 0.6.

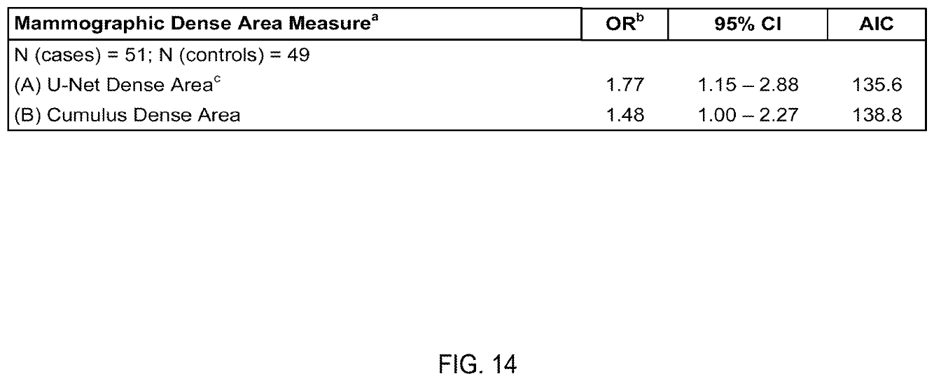

In the case-control analysis of 51 CBC cases and 49 UBC controls in the holdout set, CBC had a statistically significant association with U-net MDA (odds per adjusted standard deviation (OR)=1.77, 95% CI 1.15-2.88) after multivariable adjustment (FIG. 14). The association between CBC and Cumulus MDA was somewhat weaker and not statistically significant (OR=1.48, 95% CI 1.00-2.27). The model fit, based on Akaike's Information Criterion (AIC), was better for the U-net MDA model (AIC=135.6) compared to the Cumulus MDA model (138.8).

Disclosed herein is an accurate method to automate MDA on cranio-caudal view mammograms using a CNN algorithm based on the U-net architecture. Exceptional precision-recall and excellent Dice/Jaccard scores for overlap with the current quantitative gold standard based on CUMULUS software were observed. Moreover, in the case-control analysis, MDA estimated by the U-net algorithm had a stronger association with CBC compared to standard Cumulus MDA. This finding is particularly striking and remarkable, as the automated algorithm outperformed the method used to train the algorithm, suggesting that the U-net MDA measure identified variation in CBC risk that is not captured via simple intensity thresholding.

While previous studies have reported the use of convolutional neural networks for segmentation of biomedical images (17,18), the present disclosure is the first to evaluate the U-net architecture for estimating MDA on a large data set of mammograms of the unaffected breast, and the first explore its potential for breast cancer risk prediction. The fully convolutional design and the combination of contextual (presence of dense regions) and spatial (localization and structural) information via the skip connections are some of the key features that enable the algorithm to produce efficient and accurate image segmentations. These results demonstrate the efficacy of this approach on digitized mammograms and its subsequent use for breast cancer risk prediction. The results on digital mammograms using the model trained on digitized mammograms shows the potential for generalization to other image types, while noting the need to re-train the algorithm on the respective images to account for the shift in the data distribution.

The development of accurate, automated programs to estimate quantitative breast density are integral for risk prediction and personalization of breast cancer surveillance. However, there are several barriers to widespread clinical implementation. First, any method for automatic quantitative breast density estimation would require validation in prospective screening studies. Second, there are not yet accepted cut-offs for quantitative breast density that would be considered “high-risk,” which currently limits the clinical usefulness of a potentially accurate method, Quantra (33). It is important to develop accurate and validated estimates of quantitative breast density to reduce the uncertainty of risk prediction and, in the long-term, personalize breast screening and surveillance.

In this study, MDA measured by the U-net algorithm had a somewhat stronger association with CBC than Cumulus MDA, indicating that the U-net MDA measure disclosed herein predicts future cancer risk at least as well as the quantitative gold standard. Additionally, the spread (SD) of the U-net density was lower than that for Cumulus, potentially reflecting the absence of human variability in the assessment. These findings may indicate that the U-net algorithm has consolidated cancer-predicting components of Cumulus MDA to create a more refined measure of breast cancer risk.

These results demonstrate that a U-net based CNN architecture trained on quantitative Cumulus density estimates produced an accurate and reproducible measure of mammographic density. The automated measure had a significant association with breast cancer risk, potentially with more precision than standard Cumulus. This approach is useful for improving risk stratification for breast cancer beyond clinical breast density assessments.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Engmann N J, Golmakani M K, Miglioretti D L, Sprague B L, Kerlikowske K. Population-Attributable Risk Proportion of Clinical Risk Factors for Breast Cancer. JAMA Oncol 2017;3(9): 1228-36 doi 10.1001/jamaoncol.2016.6326.
2. Bissell M C S, Kerlikowske K, Sprague B L, Tice J A, Gard C C, Tossas K Y, et al. Breast Cancer Population Attributable Risk Proportions Associated with Body Mass Index and Breast Density by Race/Ethnicity and Menopausal Status. Cancer Epidemiology Biomarkers &amp; Prevention 2020: cebp.0358.2020 doi 10.1158/1055-9965.Epi-20-0358.
3. Alomaim W, O'Leary D, Ryan J, Rainford L, Evanoff M, Foley S. Variability of Breast Density Classification Between US and UK Radiologists. J Med Imaging Radiat Sci 2019;50(1): 53-61 doi 10.1016/j.jmir.2018.11.002.
4. Gard C C, Aiello Bowles E J, Miglioretti D L, Taplin S H, Rutter C M. Misclassification of Breast Imaging Reporting and Data System (BI-RADS) Mammographic Density and Implications for Breast Density Reporting Legislation. Breast J 2015;21(5): 481-9 doi 10.1111/tbj. 12443.
5. Byng J W, Boyd N F, Fishell E, Jong R A, Yaffe M J. The quantitative analysis of mammographic densities. Phys Med Biol 1994;39 (10): 1629-38 doi 10.1088/0031-9155/39/10/008.
6. Nguyen T L, Aung Y K, Evans C F, Yoon-Ho C, Jenkins M A, Sung J, et al. Mammographic density defined by higher than conventional brightness threshold better predicts breast cancer risk for full-field digital mammograms. Breast Cancer Res 2015; 17:142 doi 10.1186/s13058-015-0654-4.
7. Keller B M, Chen J, Daye D, Conant E F, Kontos D. Preliminary evaluation of the publicly available Laboratory for Breast Radiodensity Assessment (LIBRA) software tool: comparison of fully automated area and volumetric density measures in a case-control study with digital mammography. Breast Cancer Res 2015;17:117 doi 10.1186/s13058-015-0626-8.
8. Pérez-Benito F J, Signol F, Perez-Cortes J C, Fuster-Baggetto A, Pollan M, Pérez-Gómez B, et al. A deep learning system to obtain the optimal parameters for a threshold-based breast and dense tissue segmentation. Comput Methods Programs Biomed 2020; 195:105668 doi 10.1016/j.cmpb.2020.105668.
9. Deng J, Ma Y, Li D A, Zhao J, Liu Y, Zhang H. Classification of breast density categories based on SE-Attention neural networks. Comput Methods Programs Biomed 2020; 193:105489 doi 10.1016/j.cmpb.2020.105489.
10. Trivizakis E, Ioannidis G S, Melissianos V D, Papadakis G Z, Tsatsakis A, Spandidos D A, et al. A novel deep learning architecture outperforming ‘off-the-shelf’ transfer learning and feature-based methods in the automated assessment of mammographic breast density. Oncol Rep 2019;42(5): 2009-15 doi 10.3892/or.2019.7312.
11. Saffari N, Rashwan H A, Abdel-Nasser M, Kumar Singh V, Arenas M, Mangina E, et al. Fully Automated Breast Density Segmentation and Classification Using Deep Learning. Diagnostics (Basel) 2020;10(11) doi 10.3390/diagnostics10110988.
12. Ciritsis A, Rossi C, Vittoria De Martini I, Eberhard M, Marcon M, Becker A S, et al. Determination of mammographic breast density using a deep convolutional neural network. Br J Radiol 2019;92(1093): 20180691 doi 10.1259/bjr.20180691.
13. Shelhamer E, Long J, Darrell T. Fully Convolutional Networks for Semantic Segmentation. IEEE Trans Pattern Anal Mach Intell 2017;39(4): 640-51 doi 10.1109/tpami.2016.2572683.
14. Lehman C D, Yala A, Schuster T, Dontchos B, Bahl M, Swanson K, et al. Mammographic Breast Density Assessment Using Deep Learning: Clinical Implementation. Radiology 2019;290(1): 52-8 doi 10.1148/radiol.2018180694.
15. Haji Maghsoudi O, Gastounioti A, Scott C, Pantalone L, Wu F F, Cohen E A, et al. Deep-LIBRA: An artificial-intelligence method for robust quantification of breast density with independent validation in breast cancer risk assessment. Med Image Anal 2021;73:102138 doi 10.1016/j.media.2021.102138.
16. Kallenberg M, Petersen K, Nielsen M, Ng A Y, Pengfei D, Igel C, et al. Unsupervised Deep Learning Applied to Breast Density Segmentation and Mammographic Risk Scoring. IEEE Trans Med Imaging 2016;35(5): 1322-31 doi 10.1109/tmi.2016.2532122.
17. Fabijanska A. Segmentation of corneal endothelium images using a U-Net-based convolutional neural network. Artificial intelligence in medicine 2018;88:1-13 doi 10.1016/j.artmed.2018.04.004.
18. Zhang Y, Chen J-H, Chang K-T, Park V Y, Kim M J, Chan S, et al. Automatic Breast and Fibroglandular Tissue Segmentation in Breast MRI Using Deep Learning by a Fully-Convolutional Residual Neural Network U-Net. Academic radiology 2019;26(11): 1526-35 doi 10.1016/j.acra.2019.01.012.
19. Baccouche A, Garcia-Zapirain B, Castillo Olea C, Elmaghraby A S. Connected-UNets: a deep learning architecture for breast mass segmentation. NPJ Breast Cancer 2021;7(1): 151 doi 10.1038/s41523-021-00358-x.
20. Pi J, Qi Y, Lou M, Li X, Wang Y, Xu C, et al. FS-UNet: Mass segmentation in mammograms using an encoder-decoder architecture with feature strengthening. Comput Biol Med 2021; 137:104800 doi 10.1016/j.compbiomed.2021.104800.
21. Dalmiş M U, Litjens G, Holland K, Setio A, Mann R, Karssemeijer N, et al. Using deep learning to segment breast and fibroglandular tissue in MRI volumes. Med Phys 2017;44(2): 533-46 doi 10.1002/mp.12079.
22. Reiner A S, Sisti J, John E M, Lynch C F, Brooks J D, Mellemkjær L, et al. Breast Cancer Family History and Contralateral Breast Cancer Risk in Young Women: An Update From the Women's Environmental Cancer and Radiation Epidemiology Study. J Clin Oncol 2018;36(15): 1513-20 doi 10.1200/jco.2017.77.3424.
23. Knight J A, Blackmore K M, Fan J, Malone K E, John E M, Lynch CF, et al. The association of mammographic density with risk of contralateral breast cancer and change in density with treatment in the WECARE study. Breast Cancer Res 2018;20(1): 23 doi 10.1186/s13058-018-0948-4.
24. Otsu N. A Threshold Selection Method from Gray-Level Histograms. IEEE Transactions on Systems, Man, and Cybernetics 1979;9(1): 62-6 doi 10.1109/TSMC.1979.4310076.
25. Barthelme S. imager: Image Processing Library Based on ‘CImg’. 0.42.32020.
26. Pau G, Fuchs F, Sklyar O, Boutros M, Huber W. EBImage-an R package for image processing with applications to cellular phenotypes. Bioinformatics 2010;26(7): 979-81 doi 10.1093/bioinformatics/btq046.
27. Meyer D, Dimitriadou E, Hornik K, Weingessel A, Leisch F. e1071: Misc Functions of the Department of Statistics, Probability Statistics Group, TU Wien. 1.7-32019.
28. Drozdzal M, Vorontsov E, Chartrand G, Kadoury S, Pal C. The Importance of Skip Connections in Biomedical Image Segmentation. In: Carneiro G, Mateus D, Peter L, Bradley A, Tavares J M R S, Belagiannis V, et al., editors. Deep Learning and Data Labeling for Medical Applications. Cham: Springer International Publishing; 2016. p 179-87.
29. Goodfellow I, Bengio Y, Courville A. Deep learning. MIT press; 2016.
30. Shen X, Tian X, Liu T, Xu F, Tao D. Continuous Dropout. IEEE Transactions on Neural Networks and Learning Systems 2018;29(9): 3926-37 doi 10.1109/TNNLS.2017.2750679.
31. Kingma D P, Ba J. Adam: A Method for Stochastic Optimization. CoRR 2015; abs/1412.6980.
32. Hopper J L. Odds per Adjusted Standard Deviation: Comparing Strengths of Associations for Risk Factors Measured on Different Scales and Across Diseases and Populations. Am J Epidemiol 2015; 182(10): 863-7 doi 10.1093/aje/kwv193.
33. Richard-Davis G, Whittemore B, Disher A, Rice V M, Lenin R B, Dollins C, et al. Evaluation of Quantra Hologic Volumetric Computerized Breast Density Software in Comparison With Manual Interpretation in a Diverse Population. Breast Cancer: Basic and Clinical Research 2018; 12:1178223418759296 doi 10.1177/1178223418759296.
34. Burton A, Byrnes G, Stone J, Tamimi R M, Heine J, Vachon C, et al. Mammographic density assessed on paired raw and processed digital images and on paired screen-film and digital images across three mammography systems. Breast Cancer Res 2016;18(1): 130-doi 10.1186/s13058-016-0787-0.

1. A method of determining density values from mammograms, comprising:
identifying, by a computing system, a first mammogram of a first breast region of a first subject, the first mammogram having a first region of interest (ROI) corresponding to a first dense area of the first breast region;
applying, by the computing system, the first mammogram to a machine learning (ML) model to generate a first segmentation map identifying the first ROI within the first mammogram, the ML model established using a training dataset comprising a plurality of examples, each of the plurality of examples comprising (i) a respective second mammogram of a second breast region of a corresponding second subject; and (ii) a respective second segmentation map identifying a second ROI in the respective second mammogram corresponding to a second dense area of the second breast region;
determining, by the computing system, a density value for the first dense area of the first breast region based on the first segmentation map; and
storing, by the computing system, using one or more data structures, an association between the first subject and the density value.

2. The method of claim 1, further comprising classifying, by the computing system, the first subject into one of a plurality of risk levels each associated with a likelihood of occurrence of breast cancer based on the density value for the first dense area of the first breast region.

3. The method of claim 1, further comprising categorizing, by the computing system, the first dense area of the first breast region into one of a plurality of density types based on a ratio of a first portion of the first segmentation map corresponding to the first ROI and a second portion of the first segmentation map outside the first ROI.

4. The method of claim 1, further comprising providing, by the computing system, information for presentation based on the association between the first subject and the density value.

5. The method of claim 2, wherein the breast cancer is one of HER2-positive breast cancer, estrogen receptor-positive breast cancer, progesterone receptor-positive breast cancer, or triple negative breast cancer.

6. The method of claim 1, further comprising administering one or more of: a radiation therapy, immunotherapy, chemotherapy or surgery to the first subject, when the density value of the first subject is elevated relative to a predetermined threshold.

7. The method of claim 6, wherein the predetermined threshold is based on a plurality of density values from a corresponding plurality of control subjects without breast cancer.

8. The method of claim 1, wherein identifying further comprises receiving, from a mammograph device, the first mammogram of the first breast region of the first subject at one of (i) prior to diagnosis of breast cancer in the first subject or (ii) after treatment of the breast cancer in the first subject.

9. The method of claim 1, wherein determining the density value further comprises determining the density value based on a ratio of a first portion of the first segmentation map corresponding to the first ROI and a second portion of the first segmentation map outside the ROI.

10. The method of claim 1, wherein the density value comprises a fibroglandular density value selected from among entirely fatty, scattered fibroglandular density, heterogeneously dense, or extremely dense.

11.-20. (canceled)

21. A system for determining density values from mammograms, comprising:
a computing system having one or more processors coupled with memory, configured to:
identify a first mammogram of a first breast region of a first subject, the first mammogram having a first region of interest (ROI) corresponding to a first dense area of the first breast region;
apply the mammogram to a machine learning (ML) model to generate a first segmentation map identifying the first ROI within the mammogram, the ML model established using a training dataset comprising a plurality of examples, each of the plurality of examples comprising (i) a respective second mammogram of a second breast region of a corresponding second subject and (ii) a respective second segmentation map identifying a second ROI in the respective second mammogram corresponding to a second dense area of the second breast region;
determine a density value for the first dense area of the first breast region based on the first segmentation map; and
store, using one or more data structures, an association between the first subject and the density value.

22. The system of claim 21, wherein the computing system is further configured to classify the first subject into one of a plurality of risk levels each associated with a likelihood of occurrence of breast cancer based on the density value for the first dense area of the first breast region.

23. The system of claim 21, wherein the computing system is further configured to categorize the first dense area of the first breast region into one of a plurality of density types based on a ratio of a first portion of the first segmentation map corresponding to the first ROI and a second portion of the first segmentation map outside the first ROI.

24. The system of claim 21, wherein the computing system is further configured to provide information for presentation based on the association between the first subject and the density value.

25. The method of claim 22, wherein the breast cancer is one of HER2-positive breast cancer, estrogen receptor-positive breast cancer, progesterone receptor-positive breast cancer, or triple negative breast cancer.

26. The method of claim 21, further comprising administering one or more of: a radiation therapy, immunotherapy, chemotherapy or surgery to the first subject, when the density value of the first subject is elevated relative to a predetermined threshold.

27. The method of claim 26, wherein the predetermined threshold is based on a plurality of density values from a corresponding plurality of control subjects without breast cancer.

28. The system of claim 21, wherein the computing system is further configured to receive, from a mammograph device, the first mammogram of the first breast region of the first subject at one of (i) prior to diagnosis of breast cancer in the first subject or (ii) after treatment of the breast cancer in the first subject.

29. The system of claim 21, wherein the density value comprises a fibroglandular density value selected from among entirely fatty, scattered fibroglandular density, heterogeneously dense, or extremely dense.
